(19) United States
(12) Patent Application Publication
(10) Pub. No.: US 2007/0213694 A1
(43) Pub. Date: Sep. 13, 2007

(54) Self-contained controlled pulsed light emitter for diverse skin care and treatment and a method thereof

(76) Inventors: Paul Perl, Tel-Aviv (IL); Arriaza Francisco, Sabadell (ES)

Correspondence Address: BROWDY AND NEIMARK, P.L.L.C.;624 NINTH STREET, NW, SUITE 300, WASHINGTON, DC, 20001-5303, US

(21) Appl. No.: 11/370,161

(22) Filed: Mar. 8, 2006

(51) Int. Cl. A61B 18/18; A61N 5/06

(52) U.S. Cl. 606/9; 607/88

(57) ABSTRACT

A self-contained controlled pulsed light emitter useful for skin care, dermal applications and topical treatment, is adapted to selectively optimize broadband light spectrum via electronic manipulation, without physically changing light sources; and to maintain such spectrum regardless of the change of the light energy via time discharge. The emitter includes a light source such as flash lamp or a discharge lamp which provides a controlled pulsed light for irradiating a predetermined region of a skin to be treated; and a display adapted to select (a) skin care applications and treatment, and (b) the intensity of the output energy.

### FIELD OF THE INVENTION

The present invention generally relates to a self-contained controlled pulsed light emitter for diverse skin care and treatment. More specifically, the present invention relates to capability to selectively optimize broadband light spectrum via electronic manipulation, in order to utilize said emitter for different applications requiring different broadband spectrum without physically changing light sources.

### BACKGROUND OF THE INVENTION

A flash lamp is an electric glow discharge lamp designed to produce extremely intense, incoherent, full-spectrum white light for very short durations.

The lamp is comprised of a sealed tube, often made of fused quartz, which is filled with a mixture of gases, primarily xenon, and electrodes to carry electrical current to the gas mixture. Additionally, a high voltage power source is necessary to energize the gas mixture; this high voltage is usually stored on a capacitor so as to allow very speedy delivery of very high electrical current when the lamp is triggered.

The electrodes protrude into each end of the tube, and are connected to a capacitor that is charged to a relatively high voltage. This is usually between 100 and 2000 volts, depending on the length of the tube, and the specific gas mixture.

A flash is initiated by first ionizing the gas mixture, then sending a very large pulse of current through the ionized gas. Ionization is necessary to decrease the electrical resistance of the gas so that a pulse measuring as much as thousands of amperes traverse through the tube. The initial ionization pulse, or trigger pulse, may be applied to one of the internal electrodes, or to a metal band or wire that is wrapped around the glass tube. When the trigger pulse is applied, the gas becomes ionized, and the capacitor immediately discharges through the tube. When this current pulse traverses through the tube, it excites electrons surrounding the gas atoms causing them to jump to higher energy levels. The atoms' electrons immediately drop back to a lower orbit, producing photons in the process, which results in a “flash” or emission of high energy electromagnetic waves in the range of wave length that preferably goes from ultraviolet to infrared.

The flash that emanates from a flash lamp may be so intense, that it can ignite flammable materials within a short distance of the tube. Carbon nanotubes are particularly susceptible to this spontaneous ignition when exposed to the light from a flashtube. Similar effects may be exploited for use in aesthetic or medical procedures such as hair removal, tattoo removal, epidermis rejuvenation and destroying lesions or moles. Discharge durations for common flashlamps are in the microsecond to a few milliseconds range and can have repetition rates of hundreds of hertz. This discharge of energy is applied to the patient's skin through appropriate devices by an operator or a therapist, as a treatment.

US Pat. App. No. 2005133740 discloses a variable wavelength ultraviolet lamp and provides an apparatus for selectively producing one or more of a plurality of wavelength distributions of light. The lamp comprises a primary light source having a primary wavelength distribution, at least one wavelength-transforming material that, in response to illumination by the primary light source produces secondary light having a wavelength distribution different from the primary light wavelength distribution. The wavelength-transforming material is disposed on a substrate external to the primary light source, and a wavelength-transforming material selection mechanism for placing at least a portion of one or more selected wavelength-transforming materials in front of the primary light source, in a selected preferred direction of light emission from the apparatus, such that the selected wavelength-transforming materials emit from the apparatus light having a wavelength distribution different from the primary light wavelength distribution.

Japanese Pat. No. 9099107 discloses a method and device for electromagnetic medical treatment. A skin medical treatment apparatus is so constructed that a light source having an external glass tube is disposed at one focal point in an elliptical reflector in housing, and preferably a gas filled linear flash lamp is used as the light source. The reflector is positioned is such a manner that the cured region of the skin is at another focal point, and a spectrum, a pair of optical filters 18 for controlling the intensity of light and an iris are disposed in an opening part on the opposite side to the light source of the reflector. Light reflected on the skin is monitored by a detecting device, and according to the monitoring result, the optimum medical treatment conditions are determined by a micro-processor connected to the user interface.

Japanese Pat. No. 2226651 discloses a flash lamp discharge apparatus. A flash discharge tube of a flash discharge device has an air-tight glass envelope capable of sealing mixed gas of two kinds or more of different ionization potential. When a trigger signal is applied from a route, a switch is started, energy in a main capacitor is partly added to a coil of a booster, and voltage in a coil of size sufficient for ionizing gas of one kind in the envelope is applied to gas through an electrode. When the gas is ionized, its electric resistance is decreased; energy of the main capacitor is generated from the discharge tube as a shape of flash light having specific spectrum distribution.

A cost-effective controlled pulsed light emitter for diverse skin care and treatment and a method thereof thus meets a long felt need. Because none of these prior art references disclose a emitter that selectively optimizes broadband light spectrum via electronic manipulation, in order to utilize said emitter for different applications requiring different broadband spectrum without physically changing lamps. Also, none of the literature cited an emitter adapted to maintain such spectrum regardless of the increase of the energy of light via time discharge.

### SUMMARY OF THE INVENTION

It is thus one object of the present invention to provide an efficient self-contained controlled pulsed light emitter useful for skin care, dermal applications and topical treatment, adapted to selectively optimize broadband light spectrum via electronic manipulation, without physically changing light sources; and to maintain such spectrum regardless of the change of the light energy via time discharge; said emitter comprising: a light source such as flash lamp or a discharge lamp which provides a controlled pulsed light for irradiating a predetermined region of a skin to be treated; and, a display adapted to select (a) said care, applications and treatment and (b) the intensity of the output energy.

Another aspect of the present invention is a method for selectively optimizing broadband light spectrum via electronic manipulation for diverse skin care and treatment, without physically changing light sources by means of controlled pulsed light emitter, said method also comprising maintaining such spectrum regardless of the change of the light energy via time discharge.

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### BRIEF DESCRIPTION OF THE FIGURES

In order to understand the invention and to see how it may be implemented in practice, several embodiments will now be described, by way of non-limiting example only, with reference to the accompanying drawing, in which

FIG. 1 represents the spectral energy of a flash pulse for miscellaneous blackbody temperatures for a given output energy according to Plank's law;

FIG. 2 is a schematic diagram of the principle of the simplified power circuitry, illustrating a preferred embodiment of the device of the present invention, and,

FIG. 3 represents the spectrum of the light for two different broadband spectrums emitted by the emitter of the present invention.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a self-contained controlled pulsed light emitter for diverse skin care and treatment and method thereof.

The term ‘about’ refers hereinafter to a tolerance of ±20% of the defined measure.

The term ‘diverse skin care, applications and treatment’ refers hereinafter to any cosmetic or dermatological treatments such as hair removal or any treatment of medical disorders of the skin, including in a non limiting manner skin rejuvenation, active acne treatment, vascular and pigmented lesion.

The term ‘light emission’ refers hereinafter to any electromagnetic radiation of any wavelength, preferably the light emission lies in the range of about 500 to about 800 nm, or in the range of about 600 nm to about 1000 nm, or for infrared light in the range of about 800 nm to about 1800 nm.

The term ‘self-contained’ refers hereinafter to a single handpiece allowing multiple broadband emissions.

In a classical free discharge circuit, the current across the lamp is free and varies along the flash pulse. The light spectrum also varies during the flash pulse; moreover, the current is very high. The output light is very blue with a large UV emission with a blackbody temperature of about 8,000K to 12,000 K; the flash duration is very short, typically from 1 to a few ms.

In the present invention, the emitter is adapted for delivering a constant and controlled broadband optical light in the desired light spectrum. The obtained pulsed light increases the efficacy of the treatment by selecting the desired output energy for this given light spectrum. This system gives a constant current discharge and a stable spectral output across the entire pulse.

This system is a single handpiece allowing at least a triple broadband emission via simple switch manipulation. For example, for visible light, the spectrum emission lies in the range of about 500 to about 800 nm, or in the range of about 600 nm to about 1,000 nm, or for infrared light in the range of about 800 nm to about 1800 nm.

This system uses an efficient skin cooling to limit the heating effect to the dermis.

The applications of the system depicts in the present invention are diverse; for example with infrared emission skin tightening, collagen stimulation or a wide range of cutaneous disorders including facial rhytids, wrinkles, stretch marks, acne scars can be performed; with visible emission hair removal, pigment and vascular lesions can be treated.

This single system may include at least three different programs; e.g. option I selects a long-range wavelengths and mid-range pulse widths; option II selects a short-range wavelengths and short-range pulse widths; and, option III selects mid-range wavelengths and long-range pulse widths.

The control device for flashlamps includes a circuit that controls lamps operation so that the operator can easily determine the range of wavelengths and the amount of energy that must be emitted by the lamp, not depending on the frequency, rate and the desired time of application.

The device, duly simplified, normally includes a flash or discharge lamp, a coil and a charge condenser connected to a power supply and determining the tension to which the above mentioned flash lamp is subjected. The circuit also includes a power diode through which the discharge current passes when the lamp is triggered.

This circuit includes a mechanism of flow and rate control that stimulates and regulates the charge circuit of the lamp. A single pulse with several modulated current peaks is provided by alternating consecutive and very rapid current increase and decrease processes. In this way, while the current that circulates through the lamp is appreciably constant, the temperature of the plasma of the lamp is maintained, the light spectrum being thus constant and known. The program unit carries this process out during the time deemed necessary, for instance, between 5 ms and 240 ms, thus measuring out the desired energy. This current control is carried out between a narrow maximum and minimum threshold which optimizes the light spectral frequency by regulating the lamp output power. The required amount of energy is obtained by adjusting the duration of the pulse.

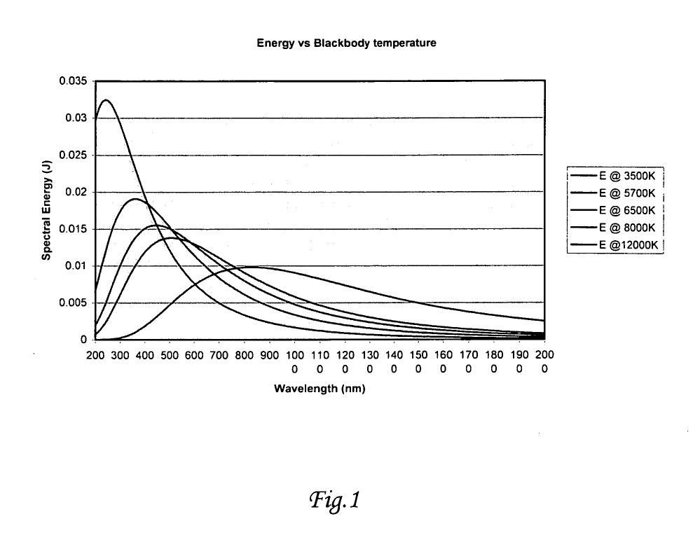

Reference is made now to FIG. 1, representing the spectral energy of a flash pulse for miscellaneous blackbody temperatures for a given output energy according to Plank's law.

The output energy for a given spectral band for example for about 500 to about 900 nm is the area under the curve from the 500 nm wavelength to the 900 nm wavelength. With a blackbody temperature of about 12,000K, a large amount of energy is wasted from about 200 to about 500 nm, and the output energy in the range of about 500 nm to about 900 nm is low. On the other hand with a temperature of about 3,500K, the output energy is mainly in the near infrared. The 5,700 K temperature gives the maximum output energy in the 500 to 900 nm bands. The same optimization can easily be achieved for another spectral band.

The blackbody temperature T being optimized, the required power density Pd within the flash lamp is calculated, according to the Stephan law: Pd=σ*T4.

The power density is the power P within the lamp divided by the internal active surface of the lamp, e.g. the inner perimeter of the lamp multiplied by the arc length. Therefore, P can be calculated from the lamp physical parameters. The power P within the lamp is equal to P=K₀*I(3/2). K₀ is the impedance of the lamp depends on the lamp geometry, gas and filling pressure. K₀ is constant for a given lamp and I is the current across the lamp. The capability of controlling the discharge current gives the capability to optimize the output spectrum of the light. The control of the discharge current optimizes the spectrum of the lamp for a given spectral band. The lamp is operating at a constant power, in watts. The output energy is adjusted by the flash pulse duration.

For example, for the hair removal mode the output energy can be adjusted between about 8 J/cm² to about 34 J/cm², for the skin remodeling mode the energy can vary between about 12 J/cm² and about 40 J/cm² and for the vascular treatment mode the energy can vary between about 20 J/cm² and about 61 J/cm².

The light source may be any suitable flash lamp or gas discharge arc lamp such as the quartz xenon flash lamp model G5109, commercially available from The Electronic Goldmine, Arizona, USA.

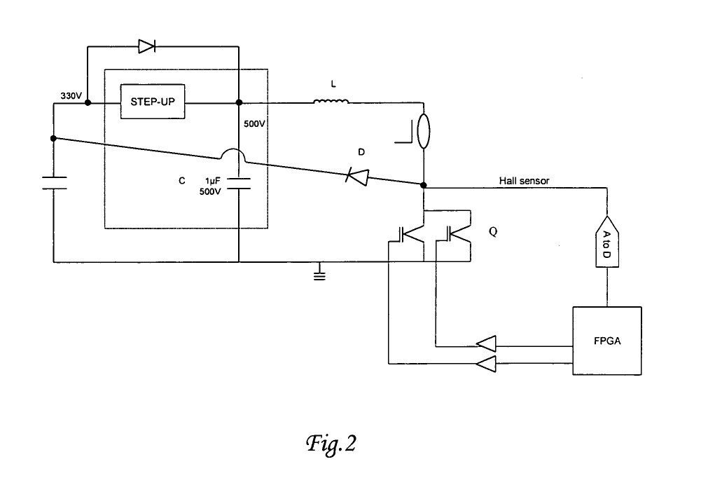

Reference is now made to FIG. 2, representing a simplified circuit illustrating a preferred embodiment of the present invention. This electronic circuit includes a charge condenser C connected to a power supply, a coil L, a flash lamp and a power diode D. The circuit converts the analog signal into a digital one. The circuit comprises a Hall Effect sensor, which is an electronic device that varies its output voltage in response to changes in magnetic field density. This sensor is a current sensing. This circuit also comprises a buffer, which is a memory used to temporarily store output or input data, a microprocessor and a field-programmable gate array (FPGA) which is a semiconductor device containing programmable logic components and programmable interconnects. At the initial state, Q is turned on. The voltage across the lamp is equal to the voltage across the main capacitor. The flash lamp is then triggered trough external triggering circuit. The current flows across L, the flash lamp and Q. When the current reaches a programmable maximum current threshold (Iₜₕ high), Q is turned off. The current flows now across L, the flash lamp and D. The current decreases until it reaches a programmable minimum current threshold (Iₜₕ low). The Q “switch” is then turned on again. This loops repeats for the required flash duration. Preferably, the duration of the polychromatic pulse is between about 5 milliseconds and about 2,500 milliseconds.

This technology has the capability of delivering a desired amount of current, with a desired discharge time according to the required application; all the applications are achieved on a single handpiece system.

This technology allows a very constant output spectrum along the flash duration; the capability to optimize the output energy for a given spectral range; longer pulses which deals better with the thermal effects. The focused, broad spectrum light is applied to the surface of the skin by way of a handpiece.

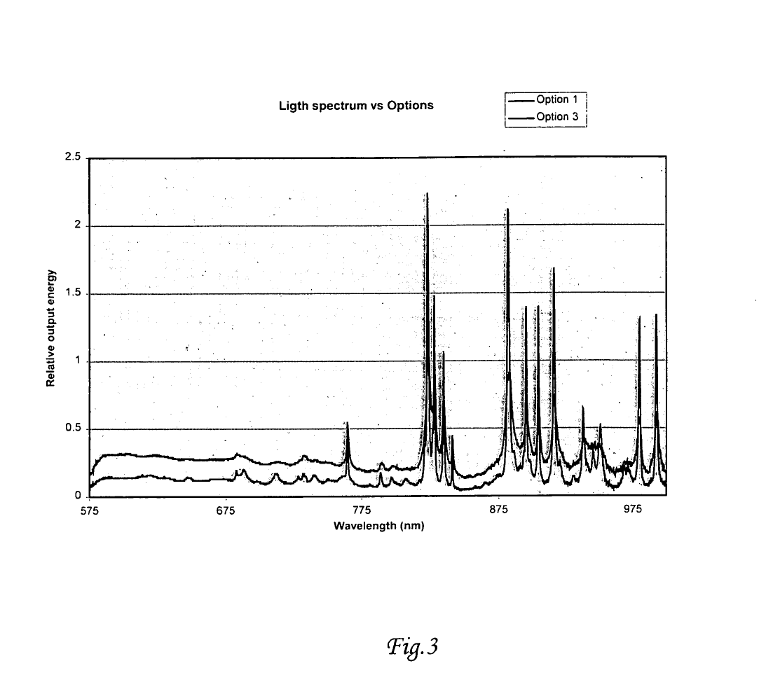

Reference is now made to FIG. 3, representing the spectrum of the light for two different broadband spectrums emitted by the emitter of the present invention. Option 1 represents short pulse emission with a blackbody temperature of 5,700 K, and Option 3 represents long pulse emission with a blackbody temperature of 3,000K.

This method additionally comprises upgrading the programmable software without changing the main unit, by means of chip memory localized in the handpiece system. This chip memory contains the different parameters of the current, time and energy changes.

1. A self-contained controlled pulsed light emitter useful for skin care, dermal applications and topical treatment, adapted to selectively optimize broadband light spectrum via electronic manipulation, without physically changing light sources; and to maintain such spectrum regardless of the change of the light energy via time discharge; said emitter comprising:
a. a light source such as flash lamp or a discharge lamp (62) which provides a controlled pulsed light for irradiating a predetermined region of a skin to be treated; and,
b. a display adapted to select (a) said care, applications and treatment and (b) the intensity of the output energy.

2. The pulsed light emitter according to claim 1, additionally comprising a control unit including a circuit that controls light sources operation, such that the operator can easily determine the range of wavelengths and the amount of energy that must be emitted by the light source, not depending on the frequency, rate and the desired time of application.

3. The pulsed light emitter according to claim 2, wherein said circuit additionally comprising:
a. at least one coil and one charge condenser connected to a power supply adapted to determine the tension to which said light source is subjected; and,
b. at least one power diode and one power switch through which said discharge current passes when the light source is triggered.

4. The pulsed light emitter according to claim 1, additionally comprising a chip memory adapted to be upgraded without changing the main unit.

5. The pulsed light emitter according to claim 1, additionally comprising an efficient skin cooling, to prevent overheating of the skin.

6. A method for selectively optimizing broadband light spectrum via electronic manipulation for diverse skin care and treatment, without physically changing light sources by means of controlled pulsed light emitter, said method also comprising maintaining such spectrum regardless of the change of the light energy via time discharge.

7. The method according to claim 5, comprising selecting optical parameters such as wavelength, energy, exposure time and rate to induce the best thermal effects in the treated skin.

8. The method according to claim 5, comprising defining calculated database before the manufacturing to optimize the output spectrum, comprising:
a. selecting the broadband spectrum according to the treatment;
b. determining the temperature of plasma that maximize the output energy, for a given spectral band;
c. calculating the required power density within the light source for an optimized temperature; and,
d. calculating the required discharge current,
such that the control of the discharge current gives the capability to optimize the output spectrum of the light.

9. The method according to claim 5, comprising setting a required discharge current across the light source, at the utilization stage, such that the current change results in a power change, which results in a temperature change and in a shift of the light spectrum.

10. The method according to claim 5, comprising providing a single pulse with several modulated current peaks by alternating consecutive and very rapid charge processes with discharge processes.

11. The method according to claim 5, comprising adjusting the output energy by the flash pulse duration when the light source is operating at a relatively constant power.

12. The method according to claim 9, wherein said output energy lies in the range of about 6 to 65 J/cm2.

13. The method according to claim 5, additionally comprising upgrading the programmable software without changing the main unit, by means of chip memory localized in the handpiece system.

14. The method according to claim 5, for vascular treatments by controlled emitting radiation of an optimized broadband spectrum of about 500 nm to about 800 nm.

15. The method according to claim 5, for hair removal by emitting radiation of an optimized broadband spectrum of about 600 nm to about 1000 nm.

16. The method according to claim 5, for skin remodeling or skin tightening by emitting radiation of an optimized broadband spectrum of about 800 nm to about 1800 nm.
